(19) United States
(12) Patent Application Publication
(10) Pub. No.: US 2006/0205077 A1
(43) Pub. Date: Sep. 14, 2006

(54) Targeted transgenesis using the rosa26 locus

(75) Inventors: Frieder Schwenk, Koln (DE); Jost Seibler, Koln (DE); Nicole Faust, Rosrath (DE); Ralf Kuhn, Koln (DE)

Correspondence Address: NORRIS, MCLAUGHLIN & MARCUS, PA, 875 THIRD AVENUE, 18TH FLOOR, NEW YORK, NY, 10022, US

(73) Assignee: Artemis Pharmaceuticals GmbH, Koln (DE)

(21) Appl. No.: 10/541,683

(22) PCT Filed: Jan. 8, 2004
(86) PCT No.: PCT/EP04/00065

§ 371 Date: Apr. 7, 2006

(30) Foreign Application Priority Data
Jan. 8, 2003 (EP) 030002497
Jan. 10, 2003 (US) 60439367

(51) Int. Cl. C12N 15/87; C12N 5/06

(52) U.S. Cl. 435/455; 435/325

(57) ABSTRACT

The invention provides a method for targeted transgenesis using the Rosa26 locus. Suitable nucleotide acid sequences and vectors for the targeted transgenesis and recombinase mediated transgenesis are provided. The Rosa26 locus proved to be a suitable integration site allowing strong and predictable expression of inserted transgenes carrying exogenous promoters.

The invention provides a method for targeted transgenesis using the Rosa26 locus. Suitable nucleotide acid sequences and vectors for the targeted transgenesis and recombinase mediated transgenesis are provided. The Rosa26 locus proved to be a suitable integration site allowing strong and predictable expression of inserted transgenes carrying exogenous promoters.

### BACKGROUND OF THE INVENTION

The generation of transgenic mice by nuclear injection of purified DNA into fertilized eggs is a widely used approach for studying gene or promoter function in vivo. However, the level and pattern of expression often varies strongly depending on copy number, configuration, and integration site of the transgene. In addition, founder mice occasionally do not transmit the transgene. Thus, a number of different founders need to be generated and tested in order to identify a useful strain, which is a laborious and time-consuming undertaking (Bradley et. al., Nature Genet., 14:121-123 (1996); Jasin et al., Proc. Natl. Acad. Sci. USA, 93:8804-8808 (1996); Dobie et al., Trends Genet., 13:127-130 (1997); Garrick et. al., Nature Genet., 18:56-59 (1998), Al-Shawi et al., Mol. Cell. Boil. 10:1192-1198 (1990)).

To overcome these limitations, homologous recombination in embryonic stem cells has been used to produce mice carrying a single copy of the transgene integrated into a predetermined site of the genome (Shaw-White et al., Transgenic Res.; (1):1-13 (1993); Bronson et al., Proc. Natl. Acad. Sci. USA, 93(17:9067-72 (1996); Hatada et al., J. Biol., Chem., 274(2):948-55 (1999); Vivian et al., Biotechniques, 27(1):154-62 (1999); Evans et al., Physiol. Genomics, March 13, 2(2):67-75 (2000); Cvetkovic et al., J. boil. Chem., 275(2):1073-8 (2000); Guillot et al., Physiol. Genomics, March 13, (2):77-83 (2000); Magness et al., Blood, 95(11):3568-77 (2000); Misra et al., BMC Biotechnol., 1(1):12 (2001); Minami et al., Blood, 100(12):4019-25 (2002); Tang et al., Genesis, 32(3):199-202 (2002)). In these studies, the ubiquitous Hprt locus was more or less successfully used for ‘targeted transgenesis’. Insertion of a lacZ gene under the control of the polyoma enhancer/HSV thymidine kinase promoter into the third exon of Hprt resulted in variable β-galactosidase expression that was both orientation and cell-type dependent (Shaw-White et al., Transgenic Res.; (1):1-13(1993)). Although transgenes under the control of the human and the chicken β-actin gene promoter resulted in widespread expression when inserted into the Hprt locus, the level of transcripts varied strongly in different tissues (Bronson et al., Proc. Natl. Acad. Sci. USA, 93(17:9067-72 (1996)). Unexpectedly, expression of these transgenes, but not of the endogenous Hprt gene appeared to be low or undetectable in kidney and liver (Bronson et al., Proc. Natl. Acad. Sci. USA, 93(17:9067-72 (1996)). Hatada et al. demonstrated that the HPRT locus suppresses the activity of both, the haptoglobin gene promoter as well as the herpes simplex thymidine kinase promoter in several tissues of mice (Hatada et al., J. Biol., Chem., 274(2):948-55 (1999)). Likewise, a human eNOS promoter-LacZ reporter gene placed in the Hprt locus was found to be inactive in hepatic vessels that otherwise express the endogenous eNOS gene (Guillot et al., Physiol. Genomics, March 13, (2):77-83 (2000). Finally, since the HPRT gene is on the X chromosome, transgene expression at this locus is subjected to random X-inactivation. The expression of the transgene in all cells of the female, therefore, requires the generation of homozygotes.

To avoid the complications referred to above, it would be desirable to define an autosomal locus that allows strong and predictable expression of transgenes inserted through homologous recombination. It is, however, not predictable for a person skilled in the art whether chromosomal loci which fulfill these criteria are available at all. Exogenous transgenes may not harbor all of the sequences necessary and sufficient for proper regulation of transcription and may therefore be influenced by cis-regulatory elements near the site of insertion.

The rosa26 locus had been identified by random insertion of retroviral sequences and a β-galactosidase-neomycin resistance fusion gene into the genome of mouse embryonic stem cells (Zambrowicz et al., Proc. Natl. Acad. Sci. USA, 94, 3789-94 (1997)). The rosa26 promoter appeared to mediate ubiquitous expression of promoter-less genes both in embryos and adult mice (Kisseberth et al., Dev. Biol., 214:128-138 (1999); Zambrowicz et al., Proc. Natl. Acad. Sci. USA, 94, 3789-94 (1997)), albeit at different levels in different organs (Vooijs et al., EMBO reports, 21:292-297 (2001)).

Moreover, WO 99/53017 describes a process for making transgenic animals which ubiquitously express a heterlogous gene, wherein the heterologous gene is under the control of a ubiquitously expressed endogenous promoter, e.g. that of the mouse Rosa26 locus. R. Dacquin et al., Dev. Dynamics 224:245-251 (2002) and K. A. Moses et al., Genesis 31:176-180 (2001) utilize the transgenic mouse strain R26R obtained according to WO 99/53017 for the expression of heterlogous genes. WO 02/098217 describes a method of targeting promoter-less selection cassettes into transcriptionally active loci, such as the Rosa26 locus.

However, a systematic comparison with other ubiquitous promoters to determine the strength of the Rosa26 promoter had not been performed. In addition, the activity of exogenous promoters inserted into the rosa26 locus has never been examined.

Finally, WO 03/020743 (published Mar. 13, 2003) describes the expression of transgenes in vivo by targeting protected transgene cassettes into predetermined loci (e.g. the Rosa26 locus), such that the introduced tissue specific exogenous promoter has at least some tissue specific activity. The protected transgene cassette contains (from 5′ to 3′ direction) a transcriptional stop signal, the exogenous tissue specific promoter and the gene of interest. The presence of a transcriptional stop signal is vital for the method of WO 03/020743 as therewith the expression pattern is determined primarily by the nature of the tissue specific exogenous promoter.

### SUMMARY OF THE INVENTION

The present invention is based on the finding that a particular chromosomal locus present within the eukaryotic genome (including that of mammalian ES cells), namely Rosa26, supports the preservation of the inherent activity of heterologous promoters inserted through homologous recombination at that locus. This chromosomal locus is therefore useful in the context of the “targeted transgenesis” approach for the efficient generation of transgenic organisms (such as mice) with a predictable transgene expression pattern.

Such a “targeted transgenesis” method comprises consecutive experimental steps. A gene expression cassette comprising a suitable promoter (e.g. a ubiquitous or tissue specific promoter, either inducible or constitutive) functionally linked to a gene of interest has to be created; subsequently a vector for the targeted insertion of the above mentioned gene expression cassette into the Rosa26 locus has to be generated; the insertion of the above mentioned gene expression cassette into the Rosa26 locus through homologous recombination or site specific recombination in embryonic stem cells follows; finally transgenic mice are generated by the injection of such genetically modified ES cells into blastocysts.

More specifically present invention provides

(1) a method for generating eukaryotic cells having a modified Rosa26 locus, which method comprises the following step (hereinafter shortly referred to as step (a)): introducing a functional DNA sequence into the Rosa26 locus of starting eukaryotic cells, preferably said functional DNA sequence is introduced into the eukaryotic cells by homologous recombination with a targeting vector comprising said functional DNA sequence flanked by DNA sequences homologous to the Rosa26 locus, or by site specific recombinase mediated recombination with a recombination vector comprising said functional DNA sequence flanked by a pair of first recombinase recognition sites (RRSs);

(2) the method of (1) above, wherein said functional DNA sequence is a gene expression cassette (a) comprising a gene of interest operatively linked to a promoter, or (b) is a DNA sequence which can be converted into such gene expression cassette;

(3) the method of (1) or (2) above, wherein the eukaryotic cells are mammalian embryonic stem (ES) cells, preferably are non-human mammalian ES cells;

(4) a targeting vector as defined in (1) or (3) above;

(5) eukaryotic cells having a modified Rosa26 locus obtainable by the method of (1) and (2) above;

(6) a method for preparing a transgenenic multi-cell organism having a modified Rosa26 locus which comprises utilizing the method as defined in (1) and (3) above;

(7) the method of (6) above, wherein the transgenenic multi-cell organism is a non-human mammal and said method comprises modifying an ES cell as defined in (3) above;

(8) a transgenic multi-cell organism and non-human mammal obtainable by the above defined methods (6) and (7), respectively; and

(9) the use of the eukaryotic cell of (5) above, the transgenic multi-cell organism of (8) above, or the transgenic non-human mammal of (8) above for gene function studies, drug development, as disease model, etc.

The method of the invention offers several advantages over the current technology of pronuclear injection. In particular, the targeting vector allows insertion of a single copy of a gene expression cassette, thus avoiding modulation of transgene expression by the arrangement of multiple copies. By choosing the autosomal Rosa26 locus as insertion site, the expression pattern of the inserted transgene in the non-human animal is predictable; random X-inactivation, and/or modulation by chromosomal position effects are avoided. This also eliminates the need to generate and analyse multiple transgenic strains for any given transgene. Finally, the Rosa26 targeting vector for the site-specific integration can be used for multiple gene expression cassettes.

### DESCRIPTION OF THE FIGURES

FIG. 1: Targeted insertion of CreER and CAGGS-Cre-ER into the Rosa26 locus. A cassette comprising a Cre-ER operationally linked to a CAGGS promoter or a cassette comprising a splice acceptor site (SA) linked to a Cre-ER are inserted into the Rosa26 locus via homologous recombination. A perpendicular dash marks the insertion point within the Rosa26 locus and the rectangular boxes delinate the starting and end points of the Rosa26 transcript.

FIG. 2: Southern Blot analysis of the inducible recombination of the Rosa (reporter). (A) Genomic DNA was isolated from liver (Li) spleen (Sp) and small intestine (Si) of transgenic mice carrying the SA-creER/Rosa-rep insert or the CAGGS-creER/Rosa-rep insert. To induce the Cre-ER recombinase the mice were treated with Tamoxifen (treated). As a control, a group of mice with the SA-creER/Rosa-rep insert was left untreated (untreated). Presence of the reporter band (floxed) and deletion (deleted) of it upon an induced recombination event are indicated. (B) Transgenic mice carrying at one Rosa26 locus, a loxP flanked DNA polymerase β gene segment (pol^{βflox}) and at the other a SA-creER/Rosa-rep were treated with Tamoxifen (treated). A control group of mice was left untreated (untreated). Genomic DNA from liver (Li), spleen (Sp), kidney (Ki), heart (He), lung (Lu), thymus (Th), muscle (Mu), small intestine (Si) and brain (Br) was analysed for presence of pol^{βflox}. In a non-recombination event the pol^{βflox} band remained (floxed), in a recombination event deletion occurred (deleted). (C) As (B), but mice carried instead of the SA-creER/Rosa-rep the CAGGS-creER/Rosa-rep insert.

FIG. 3: Western Blot analysis of recombinase and α-actin expression. Proteins were extracted from rosa (SA-CreER^{T2}), and rosa (CAGGS-CreER^{T2}) mice and analyzed as described in the “Materials and Method” section. The positions of bands representing CreER^{T2} and actin are indicated. FA: fat tissue, Ty: Thymus; Sp: spleen, Br: Brain, Lu: lung, He: heart.

FIG. 4: Fabp-Cre targeting vector. An expression cassette, in which the Cre recombinase is expressed under the control of the Fabpl^{4× at -132} promoter is inserted into the Rosa26 targeting vector. This vector was used to insert the Fabp-Cre cassette into the Rosa26 locus by homologous recombination in ES cells.

FIG. 5: ROSA26 locus of the Cre reporter mice carrying a Cre substrate reporter construct. A recombination substrate (Seq ID NO: 9) has been inserted in the ROSA26 locus. The substrate consists of a CAGGS promoter followed by a cassette consisting of the hygromycin resistance gene driven by a PGK promoter and flanked by loxP recombination sites. This cassette is followed by the coding region for beta-galactosidase, which is only expressed when the hygromycin resistance gene has been deleted by recombination.

FIG. 6: In situ detection of beta-galactosidase in cryosections of different tissues of Fabp-Cre/reporter substrate double transgenic mice. Mouse tissues were embedded in OCT, frozen and cut into microsections. The sections were stained for beta-galactosidase activity (indicated by the blue color) by X-gal staining, counterstained with Nuclear Fast Red Solution, dehydrated, mounted and photographed.

### DETAILED DESCRIPTION OF THE INVENTION

The term “living organisms” according to the present invention relates to multi-cell organisms which can be vertebrates such as mammals (e.g. non-human animals such as rodents including mice and rats; and humans) or non-mammals (e.g. fish) or can be invertebrates such as insects or worms, or can be plants (higher plants, algi or fungi). Most preferred living organisms are mice and fish.

“Eukaryotic cells” and “starting eukaryotic cells” according to the present invention include cells isolated (derived) from the above defined living organisms and cultured in vitro. These cells can be transformed (immortalized) or untransformed (directly derived from living organisms; primary cell culture). The term “eukaryotic cells” also includes mono-cellular eukaryotic cells such as yeasts, etc.

It is preferred in the method (1) of the present invention that the eukaryotic cells are derived from a multi-cell organism including vertebrates, invertebrates and plants, preferably is a vertebrate cell, more preferably is derived from a mammal, including rodents such as mouse, rat, etc., or a fish such as zebrafish.

In the method (1) of the invention it is preferred that the functional DNA sequence comprises a gene encoding a protein/peptide of interest (i.e. is a expressible and translatable DNA sequence), more preferably said functional DNA sequence is a gene expression cassette (a) comprising a gene of interest operatively linked to a promoter, or (b) is a DNA sequence which can be converted into such gene expression cassette (i.e. into an operatively linked “promoter-gene of interest” construct, e.g. by subsequent modification reactions after its integration). The gene of interest within the gene expression cassette can be any gene coding for a certain protein/peptide of interest, including, but not limited to, recombinases, reporter genes, receptors, signaling molecules, transcription factors, pharmaceutically active proteins and peptides, drug target candidates, disease causing gene products, toxins, etc.

The promoter of the gene expression cassette (which is a heterologous promoter relative to the Rosa26 locus) preferably is a ubiquitous or tissue specific promoter, either constitutive or inducible. The ubiquitous promoter in the vector according to the invention is preferably selected from polymerases I, II and III dependent promoters, preferably is a polymerase II or III dependent promoter including, but not limited to, a CMV promoter, a CAGGS promoter, a snRNA promoter such as U6, a RNAse P RNA promoter such as H1, a tRNA promoter, a 7SL RNA promoter, a 5 S rRNA promoter, etc. Particularly preferred ubiquitous promoters are CAGGS, hCMV, PGK. Preferred tissue specific promoters are FABP (Saam & Gordon, J. Biol. Chem., 274:38071-38082 (1999)), Lck (Orban et al., Proc. Natl. Acad. Sci. USA, 89:6861-5 (1992)), CamKII (Tsien et al., Cell 87: 1317-1326 (1996)), CD19 (Rickert et al., Nucleic Acids Res. 25:1317-1318 (1997)); Keratin (Li et al., Development, 128:675-88 (201)), Albumin (Postic & Magnuson, Genesis, 26:149-150 (2000)), aP2(Barlow et al., Nucleic Acids Res., 25 (1997)), Insulin (Ray et al., Int. J. Pancreatol. 25:157-63 (1999)), MCK (Brüning et al., Molecular Cell 2:559-569 (1998)), MyHC (Agak et al., J. Clin. Invest., 100:169-179 (1997), WAP (Utomo et al., Nat. Biotechnol. 17:1091-1096 (1999)), Col2A (Ovchinnikov et al., Genesis, 26:145-146 (2000)); preferred inducible promoter systes are Mx (Kühn et al. Scinence, 269: 1427-1429 (1995)), tet (Urlinger et al., Proc. Natl. Acad. Sci. USA, 97:7963-8 (2000)), Trex (Feng and Erikson, Human Gene Therapy, 10:419-27). Suitable inducible promoters are the above-mentioned promoters containing an operator sequence including, but not limited to, tet, Gal4, lac, etc.

The targeting vector, recombination vector, functional DNA sequence or gene expression cassette may further comprises one ore more additional functional sequences including but not limited to (selectable) marker genes (such as the neomycin phosphotransferase gene of E. coli transposon, etc.), recombinase recognition sites (which in case of the recombination vector differ from the first recombinase recognition sites and which include loxP, FRT, variants thereof, etc.), poly A signals (such as synthetic polyadenylation sites, or the polyadenylation site of human growth hormones, etc.), splice acceptor sequences (such as a splice acceptor of adenovirus, etc.), introns, tags for protein detection, enhancers, selection markers, etc.

In a preferred embodiment methods (1) to (3) of the invention comprise homologous recombination. It is then preferred that the DNA sequences homologous to the Rosa26 locus are 0.2 to 20 kB, preferably 1 to 10 kB long. In a particularly preferred embodiment of the method (2) the eukaryotic cells are derived from mouse, the DNA sequences homologous to the Rosa26 locus are derived from the 5′ and 3′, flanking, arm of the mouse Rosa26 locus, preferably said homologous DNA sequences having the sequences shown in SEQ ID NO:4 and 5, respectively, and the promoter is a CAGGS-promoter, most preferably the targeting vector has the sequence shown in SEQ ID NO:7.

In a further preferred embodiment, methods (1) to (3) of the invention comprise recombinase mediated recombination. The insertion of transgenes or DNA segments into the genome can be mediated by site specific recombination (Fukushige & Sauer, Proc. Natl. Acad. Sci. USA 89(17):7905-9 (1992)). A site specific recombinase like cre or FLP recombines two recognition target sites like loxP or FRT, respectively. The use of two incompatible recognition target sites (F3 or F5, Schlake & Bode, Biochemistry, 1994 Nov. 1, 33(43):12746-51) or inverted recognition target sites (Feng et al., J. Mol. Biol. 292(4):779-85 (1999)) allows the insertion of DNA segments flanked by two incompatible or inverted target sites. This exchange system has been called recombinase mediated cassette exchange (RMCE). In a preferred embodiment a FLP based RMCE system is inserted into the Rosa26 locus. Said recombinase mediated recombination preferably comprises the steps:
(a1) introducing into the starting cells an acceptor DNA which integrates into the genome of the starting cell, the acceptor DNA comprising two mutally incompatible first RRSs, and introducing into the therewith obtained cell
(a2) a donor DNA comprising the same two mutually incompatible first RRSs contained in the acceptor DNA by utilizing a recombination vector as defined above; and
(a3) the recombinase which catalyzes recombination between the RRSs of the acceptor and donor.

In said recombinase mediated recombination method it is preferred that
(i) the RRS are loxP or FRT sites or variants thereof (such as single mutant recognition sited lox66 and lox71 (Albert et al., The Plant J. 7:649-659 (1995)); and/or
(ii) the acceptor DNA comprises a negative selectable marker (e.g. herpes simplex virus thymidin kinase gene, etc.) and or
(iii) the donor DNA comprises an inactive positive selection marker (e.g. neomycin phosphotransferase, etc.).

For further selectable markers it is referred to U.S. Pat. Nos. 5,487,932 and 5,464,763 which are hereby incorporated in their entirety.

In a particularly preferred embodiment of the methods (1) to (3), and in particular if the method comprises homologous recombination, the expression cassette
(i) is free of a transcriptional stop signal 5′ to the (heterologous) promoter of the cassette (i.e. is a non-protected cassette); and/or
(ii) the exogenous promoter is a ubiquitous (constitutive or inducible) promoter.

The methods (1) to (3) may further (besides step (a) defined above) comprise one or more of the steps (b) isolating the eukaryotic cells, preferably the ES cells having the desired fuctional DNA sequence integrated into the Rosa26 locus; and/or (c) modifying the integrated functional DNA sequence and isolating (ES) cells having the desired modified functional DNA sequence.

The steps (a) and (b) of the methods (1) to (3) are preferably performed in vitro. The step (c) may be performed in vitro and in vivo.

The invention also provides a method for preparing a transgenenic multi-cell organism having a modified Rosa26 locus which comprises utilizing the method as defined in (1) to (3) above. This includes a method for preparing a non-human mammal comprising modifying starting ES cells according to steps (a) to (c). The ES cells may subsequently processed according one or more of the following steps:

(d) the ES cells obtained in steps (b) or (c) are injected into blastocysts; and or

(e) transgenic non-human animals carrying one or more functional genes of interest at the Rosa26 locus are generated (viz, by well known breeding procedures).

The transgenic multi-cell organisms and non-human mammals obtainable by the method (6) and (7), respectively; preferably have an operatively functional gene expression cassette (as defined above) integrated into its Rosa26 locus. Such transgenic multi-cell organisms and non-human mammals are suitable for gene function studies, drug development, as disease model animals, etc.

The invention is further explained by the following examples and the attached figures, which are, however not to be construed so as to limit the invention.

### EXAMPLES

Materials and Methods

Plasmid Construction:

1. CreER Rosa-targeting vector: A 129 SV/EV-BAC library (Incyte Genomics) was screened with a probe against exon2 of the Rosa26 locus (amplified from mouse genomic DNA using Rscreen1s (GACAGGACAGTGCTTGTTTAAGG) (SEQ ID NO:1) and Rscreen1as (TGACTACACAATATTGCTCGCAC) (SEQ ID NO:2)). Out of the identified BACclone a 11 kb EcoRV subfragment was inserted into the HindII site of pBS. Two fragments (a 1 kb SacII/XbaI- and a 4 kb XbaI-fragment) were used as homology arms and inserted into a vector containing a FRT-flanked neomycin resistance gene (unpublished) to generate the basic Rosa26 targeting vector. The CAGGS-promoter (SEQ ID NO:6, nucleotides 1-1616) or a splice acceptor site (SA) from adenovirus (Friedrich G., Soriano P., Genes Dev., 5:1513-23 (1991)) were inserted between the 5′ arm and the FRT flanked neomycin resistance gene. The CreER^{T2} and a polyadenylation site (pA; SEQ ID NO: 6, nucleotides 3921-4099) were cloned 3′ of the SA or the CAGGS-promoter. The vector is free of a transcriptional stop sequence 5′ to the CAGGS-promoter

2. FABP-Cre Rosa-targeting vector (SEQ ID NO:8): The splice accetpr site from adenovirus (SEQ ID NO:8, nucleotides 18569-18689) was inserted into the basic Rosa26 targeting vector described in 1, above. Into the SwaI and AscI restriction sites of the resulting plasmid was inserted a 3195 bp Xba_{blunt}/AscI DNA fragment comprising in 5′ to 3′ order the polyadenylation signal from the human growth hormone gene (SEQ ID NO:8; nucleotides 18760-688; Bond et al, Science 289:1942-1946 (2000)), a modified Fabpl promoter (SEQ ID NO:8, nucleotides 702-1481; Fabpl^{4× at -132}; Simon et al., J. Biol. Chem. 272:10652-10663 (1997)), a synthetic intron (SEQ ID NO:8, nucleotides 1521-1758), the Cre coding sequence (SEQ ID NO:8, nucleotides 1778-2830) and a synthetic polyA signal (SEQ ID NO: 8, nucleotides 2888-3066).

Cell culture: Culture a and targeted mutagenesis of ES cells were carried out as previously described (Hogan et al., (Cold Spring Harbor Laboratory Press, Cold Spring Harbor N.Y.), pp. 253-289.) with ES cell lines derived from both inbred and F1 embryos.

Mice: All mice were kept in the animal facility at Artemis Pharmaceuticals GmbH in microisolator cages (Tecniplast Sealsave). B6D2F1 Mice for the generation of tetraploid blastocysts were obtained from Janvier. The polb^{flox}/rosa(CreER^{T2}) and ect2^{flox}/rosa(CreER^{T2}) mice were generated by breeding of rosa(CreER^{T2}) ES mice with βT14 (Gu et al., Science, 265, 103-106.), respectively.

Production of ES mice by tetraploid embryo complementation: The production of mice by tetraploid embryo complementation was essentially performed as described (Eggan et al., Proc Natl Acad Sci USA, 98, 6209-6214.).

Ligand administration: 100 mg Tamoxifen-free base (Sigma, T5648) was suspended in 100 μl Ethanol and solved in 1 ml sunflower oil (Sigma). This 10 mg/100 μl tamoxifen solution was, sonicated for 1-2 minutes and then stored at −20° C. For p.o. administration the solution was thawed at 55° C. and administrated to 4-8 week old mice by a feeding needle (FST Fine Science Tools GmbH, 18061-20).

Western blot analysis: Western blot analysis was performed using SDS-PAGE (NuPAGE, Invitrogen) and the Breeze Immunodetection System (Invitrogen) according to the manufacturer protocols. Immunodetection was done using sc-543 (HC-20, Santa Cruz Biotechnology, Inc.) against ER, PRB-106C against cre, actin sc-1616 Actin (I-19) against actin and rabbit polyclonal IgG (Santa Cruz Biotechnology, Inc.) antibodies.

X-Gal staining on tissue sections: To detect beta-galactosidase activity, tissues were embedded in Tissue Tec OCT (Sakura Finetek Europe B. V., The Netherlands), frozen on dry ice and cut into microsections. The sections were mounted onto slides and dried for 1-4 hours at room temperature. Sections were fixed for 5 min at room temperature in fixing solution (0,2% glutaraldehyde, 5 mM EGTA, 2 mM MgCl₂ in 0.1 M PB ((0.1 M K₂HPO₄, pH 7.3)) and washed three times for 15 min at room temperature in washing buffer (2 mM MgCl₂, 0.02% Nonidet-40 in 0.1 M PB). Subsequently, tissues were stained for beta-galactosidase activity over night at 37° C. using X-Gal solution (0.6 mg/ml X-Gal (predissolved in DMSO), 5 mM potassium hexacyanoferrat III, 5 mM potassium hexacyanoferrat II, in washing buffer). Sections were washed twice for 5 min at room temperature in PBS, counterstained with Nuclear Fast Red Solution for 10 min, rinsed shortly in aqua dest., dehydrated through a graded ethanol series and mounted in Eukitt (Sigma, Germany).

### Example 1

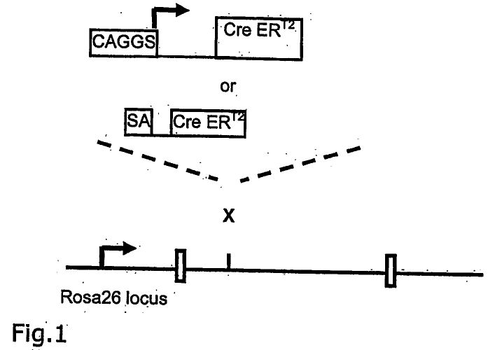

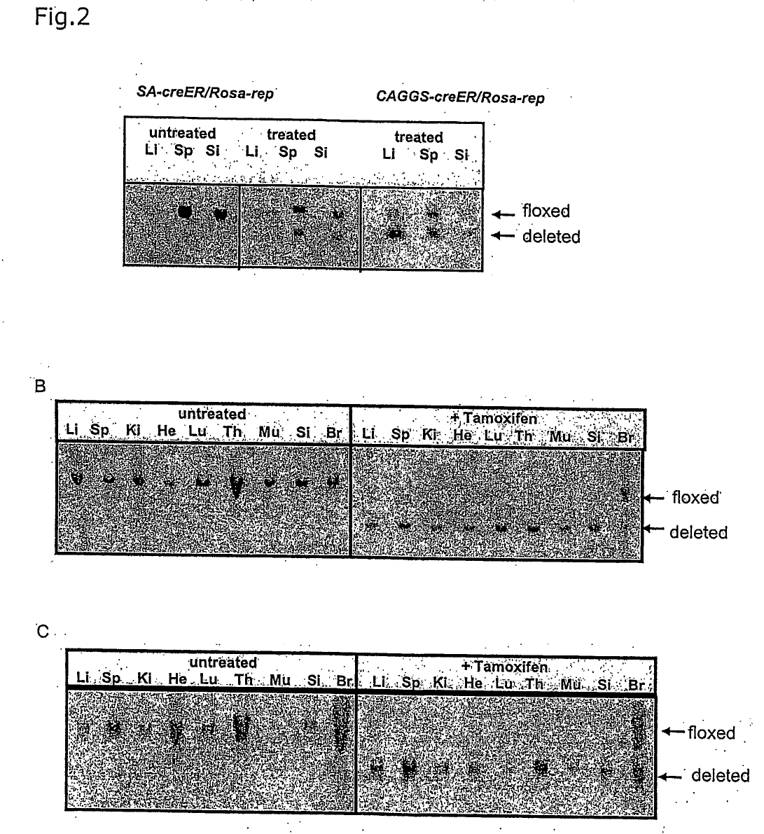

A CreER^{T2} gene (Feil et al., (1997) Biochem Biophys Res Commun., 237, 752-757) under the control of the CAGGS-promoter (Okabe, Fabs Letters 407:313-19 (1997)) was inserted into the rosa26 locus by homologous recombination in ES cells by utilizing the CreER Rosa-targeting vector as described above (FIG. 1). In addition to the CreER^{T2} gene a splice acceptor sequence (Friedrich and Soziano (1991), Genes Dev., 9, 1513-1523) was introduced as a control for the endogenous activity of the rosa26 gene promoter (FIG. 1). A loxP-flanked hygromycin resistance gene was introduced into the second allele of rosa26 to provide test substrate for Cre ER^{T2} (Seibler et al., Nucl. Acids. Res. Feb. 15, 2003, 31(4):(12) (2003)), in press). ES cells modified at both rosa26 alleles were injected into tetraploid blastocysts and completely ES cell derived mice were generated (Eggan et al., (2001). PNAS, 98, 6209-6214). Rosa(SA-CreER^{T2}/reporter) and Rosa(CAGGS-CreER^{T2}/reporter) mice were fed with daily 5 mg Tamoxifen for 5 days and recombination of the reporter was analyzed 3 days after the last administration. Southern analysis of genomic DNA from different organs showed up to 50% recombination in the Rosa(SA-CreER^{T2}/reporter) mice and up to 90% recombination in the rosa(CAGGS-CreER^{T2}/reporter) mice, respectively (FIG. 2A). As the second substrate, we used the loxP flanked DNA polymerase β gene segment (polβ^{flox}) (Gu et al., (1994). Science, 265, 103-106). The polβ^{flox}/rosa(SA-CreER^{T2}) and polβ^{flox}/rosa(CAGGS-CreER^{T2} mice were fed with 5 mg tamoxifen per day for 5 days and analyzed 3 days later. Southern blot analysis revealed that the loxP-flanked polymerase β gene segment was excised in more than 90% of cells in all organs except brain in the rosa(SA-CreER^{T2}/reporter) mice (FIG. 2B). In contrast, the degree of inducible recombination was significantly higher in rosa(CAGGS-CreER^{T2}/reporter) mice, reaching 100% efficiency in most organs and up to 70% in brain.

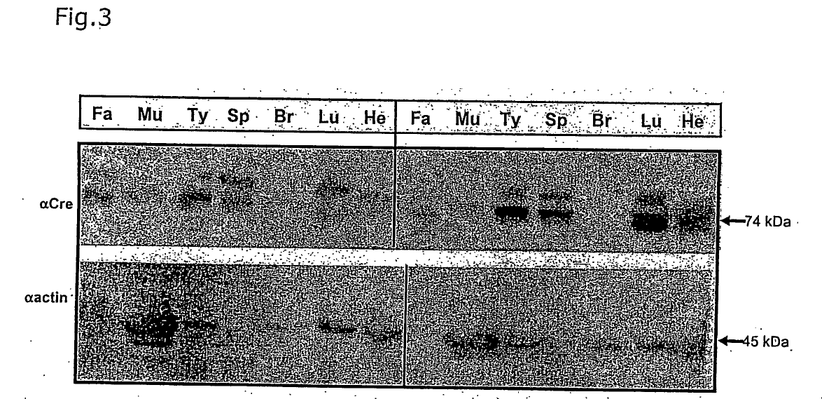

To investigate the pattern and level of CreER^{T2} expression in rosa(SA-CreER^{T2}) and rosa(CAGGS-CreER^{T2}) mice, we performed Western analysis using antibodies specific for Cre. The 74 kDa band corresponding to the CreER^{T2} fusion protein was detectable in all organs of rosa(CAGGS-CreER^{T2}) mice, including brain (FIG. 3). In contrast, the CreER^{T2} expression level in rosa(SA-CreER^{T2}) mice was significantly lower compared to the rosa(CAGGS-CreER^{T2}) strain and appeared to be undetectable in brain (FIG. 3).

### Example 2

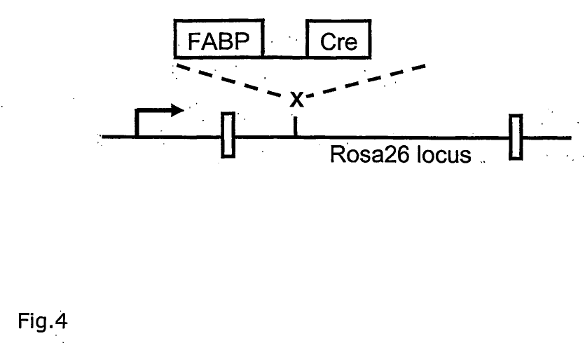

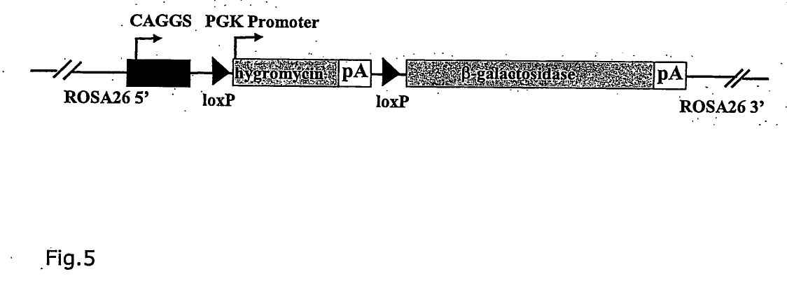

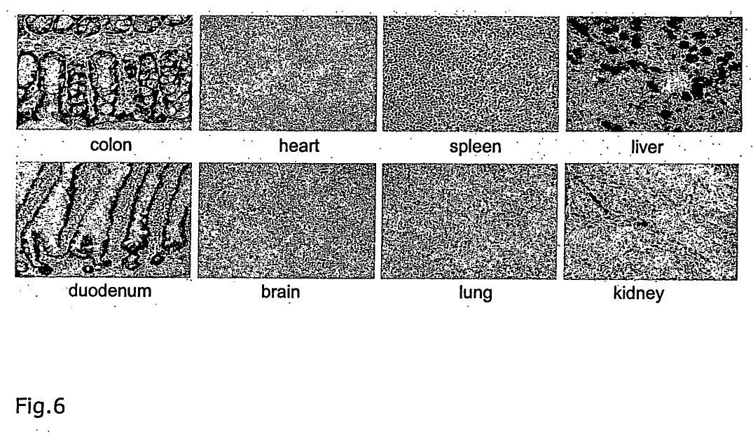

A Cre gene under the control of the Fabpl^{4× at -132}-promoter (SEQ ID NO:8; FIG. 4) was inserted into the Rosa26 locus by homologous recombination in F1 ES cells carrying a Cre reporter substrate in the second Rosa26 allele. LacZ expression from the reporter construct (SEQ ID NO:9; FIG. 5) is activated upon Cre-mediated recombination. Targeted ES cells were injected into tetraploid blastocysts to generate FABP-Cre/reporter-substrate double transgenic ES mice. The Cre recombination pattern in these mice was examined by analyzing beta-galactosidase activity in tissues sections (FIG. 6). Cre-mediated recombination in these mice was restricted to the intestinal epithelium, liver and part of the cells in the epithelium of the tubuli in the kidney, thus exactly reflecting the expression pattern of the endogenous Fabpl gene (Simon et al., J. Biol. Chem., 272:10652-10663 (1997)).

1-16. (canceled)

17. Method for generating transgenic eukaryotic cells having a modified Rosa26 locus, which method comprises (a) introducing a functional DNA sequence into the Rosa26 locus of starting eukaryotic cells, wherein said functional DNA sequence is a gene expression cassette comprising a gene of interest operatively linked to a promoter or is a DNA sequence which can be converted into such gene expression cassette.

18. The method of claim 17, wherein the functional DNA sequence is introduced into the eukaryotic cells by homologous recombination with a targeting vector comprising said functional DNA sequence flanked by DNA sequences homologous to the Rosa26 locus.

19. The method of claim 17, wherein the functional DNA sequence is introduced into the eukaryotic cells by site specific recombinase mediated recombination with a recombination vector comprising said functional DNA sequence flanked by a pair of first recombinase recognition sites (RRSs).

20. The method of claim 17, wherein the eukaryotic cells are derived from a multi-cell organism.

21. The method of claim 20, wherein the multi-cell organism is selected from the group consisting of vertebrates, invertebrates and plants.

22. The method of claim 20, wherein the eukaryotic cells are vertebrate cells.

23. The method of claim 20, wherein the eukaryotic cells are derived from a mammal.

24. The method of claim 23, wherein the mammal is a rodent.

25. The method of claim 24, wherein the rodent is selected from the group consisting of mouse and rat.

26. The method of claim 20, wherein the eukaryotic cells are derived from a fish.

27. The method of claim 26, wherein the fish is zebrafish.

28. The method of claim 20, wherein the eukaryotic cells are selected from the group consisting of primary cells and immortalized cells.

29. The method of claim 20, wherein the eukaryotic cells are mammalian embryonic stem (ES) cells.

30. The method of claim 17, wherein the gene of interest is selected from the group of genes consisting of recombinases, reporter genes, receptors, signaling molecules, transcription factors, pharmaceutically active proteins and peptides, drug target candidates, disease causing gene products and toxins.

31. The method of claim 17, wherein the promoter is a heterologous promoter.

32. The method of claim 31, wherein the promoter is selected from the group consisting of a constitutive ubiquitous promoter, a constitutive tissue specific promoter, an inducible ubiquitous promoter and an inducible tissue specific promoter.

33. The method of claim 31, wherein the promoter is selected from the group consisting of a CAGGS, hCMV, PGK, FABP, Lck, CamKII, CD19, Keratin, Albumin, aP2, Insulin, MCK, MyHC, WAP, Col2A, Mx, tet and Trex promoter.

34. The method of claim 17, wherein the functional DNA sequence or gene expression cassette further comprises one or more additional functional sequences selected from the group consisting of marker genes, second recombinase recognition sites differing from the first recombinase recognition sites, poly A signal and introns.

35. The method of claim 17, wherein the targeting vector and recombination vector further comprises functional sequences selected from the group consisting of tags for protein detection, enhancers and selection markers.

36. The method of claim 18, wherein the DNA sequences homologous to the Rosa26 locus are 0.2 to 20 kB long.

37. The method of claim 36, wherein the DNA sequences homologous to the Rosa26 locus are 1 to 10 kB long.

38. The method of claim 36, wherein the transgenic eukaryotic cells are derived from mouse and the DNA sequences homologous to the Rosa26 locus are derived from the 5′ and 3′ flanking arm of the mouse Rosa26 locus.

39. The method of claim 38, wherein said homologous DNA sequences have the sequences shown in SEQ ID NO:4 and 5, respectively.

40. The method of claim 36, wherein the transgenic eukaryotic cells are derived from mouse, and the promoter is a CAGGS-promoter.

41. The method of claim 36, wherein the targeting vector has the sequence shown in SEQ ID NO:7.

42. The method of claim 19, which comprises the steps of
(a1) introducing into the starting cells an acceptor DNA which integrates into the genome of the starting cell, the acceptor DNA comprising two mutally incompatible first RRSs, and introducing into the therewith obtained cell
(a2) a donor DNA comprising the same two mutually incompatible first RRSs contained in the acceptor DNA by utilizing a recombination vector as defined in claim 19; and
(a3) the recombinase which catalyzes recombination between the RRSs of the acceptor and donor.

43. The method of claim 42, wherein the RRS are loxP or FRT sites or variants thereof.

44. The method of claim 42, wherein the acceptor DNA comprises a negatively selectable marker gene.

45. The method of claim 42, wherein the donor DNA comprises an inactive positive selection marker.

46. The method of claim 17, which further comprises one or more of the steps selected from the group consisting of
(b) isolating the eukaryotic cells, preferably the ES cells having the desired functional DNA sequence integrated into the Rosa26 locus; and
(c) modifying the integrated functional DNA sequence and isolating ES cells having the desired modified functional DNA sequence.

47. A targeting vector comprising a functional DNA sequence as defined in claim 17.

48. A eukaryotic cell having a modified Rosa26 locus obtainable by the method of claim 17.

49. A method for preparing a transgenic multi-cell organism having a modified Rosa26 locus which comprises utilizing the method as defined in claim 17.

50. The method of claim 49, wherein the transgenic multi-cell organism is a non-human mammal and said method comprises modifying an ES cell.

51. The method of claim 49, which further comprises one or more of the steps selected from the group consisting of
(d) injecting ES cells obtained in steps (b) or (c) into blastocysts; and
(e) generating transgenic non-human animals carrying one or more functional genes of interest at the Rosa26 locus.

52. A transgenic multi-cell organism or a transgenic non-human mammal obtainable by the method of claim 49, and having an operatively functional gene expression cassette integrated into its Rosa26 locus.

53. A method for studying gene functions which comprises utilizing a biological entity selected from a eukaryotic cell, a transgenic multi-cell organism, or a transgenic non-human mammal obtainable utilizing the method of claim 17.

54. A method for drug development, which comprises contacting the drug candidate with a biological entity selected from a eukaryotic cell, a transgenic multi-cell organism, or a transgenic non-human mammal obtainable utilizing the method of claim 17.

55. A method for drug development, which comprises utilizing a biological entity selected from a eukaryotic cell, a transgenic multi-cell organism, or a transgenic non-human mammal obtainable utilizing the method of claim 17 as a disease model animal.


SEQUENCE LISTING (9 sequences)

SEQ ID NO: 1 - DNA, 23 bp, Artificial Sequence
  Description of Artificial Sequence primer Rscreen1s
gacaggacagtgcttgtttaagg

SEQ ID NO: 2 - DNA, 23 bp, Artificial Sequence
  Description of Artificial Sequence primer Rscreen1as
tgactacacaatattgctcgcac

SEQ ID NO: 3 - DNA, 13139 bp, Mus musculus
  Description Rosa26 locus
aagcttctcacgtagcaaccagagctccagagccagcagctgctgccgccttgtatactcactcctgtgatccaacacaggagcaaccttttctttaccccacccccacttcttaacacacttttttttggggggggggggggaacaagtgctccatgctggaaggattggaactatgcttttagaaaggaacaatcctaaggtcacttttaaattgaggtctttgatttgaaaatcaacaaataccaaattccaaatattcgttttaattaaaccagcaatgtggatataagcattaagttttagttttaaaaaggtcaattttccaaacattcagcaatcatatttaaatttacagctaggaacaagagccttgggtcatgtcctaccaaagaacataactcaatattctacacatgacaatctgaataaccttaaagcctctaatcccataacaggccacaaattttggacagagaactaatgatcctcctgagaaaactggaagaaatccagggaaaagaaattcctgtgtcctccaaactcagaaatctctaattatgtcagtattctctgctttagtcctaggtcagattgcacacatctaaaataacctcttaaagttttcctcctagcgacctaaaccattattaatatcaaattaaccatcaaaacactttcctctcaatatgctgcacacaaacctcctcctggaacctcctccatctggatcctccccaatcaaaagtataggtatttaacatataagcaaggaagtaatgtaaacatgaccttggtcacaaatatgtcatctaaaaacaatttagtcaaggtatggaggaaattcgagaacctgaatctttttaagtattttgagcacaggaacaattggcaaaaggaatccaggtatagacaaaacccagagcccagagctctgggcgaaaaatgagttgctggtgaagacgttacacaagtaacatgagaaagcagaaaatgcaggtcatccacgcacccctgacccaggccagcagggcgggctgcagcatcagtacacaggagaaagatccttattcctaagaatgagaaaggcaaaggcgcccgatagaataaattagcatagaaggggctttcccaggagttaaaactttccttctgagcgattacctactaaaaccagggcttttgcccactaccatttacctaggatcttggcttgcacggattcataggggcatatccctccccctcttctttagagtcgttcttaaaagatcgctctccacgccctaggcagggaaaacgacaaaatctggctcaattccaggctagaaccctacaaattcaacagggatatcgcaaggatactggggcatacgccacagggagtccaagaatgtgaggtgggggtggcgaaggtaatgtctttggtgtgggaaaagcagcagccatctgagataggaactggaaaaccagaggagaggcgttcaggaagattatggaggggaggactgggcccccacgagcgaccagagttgtcacaaggccgcaagaacaggggaggtggggggctcagggacagaaaaaaaagtatgtgtattttgagagcagggttgggaggcctctcctgaaaagggtataaacgtggagtaggcaatacccaggcaaaaaggggagaccagagtagggggaggggaagagtcctgacccagggaagacattaaaaaggtagtggggtcgactagatgaaggagagcctttctctctgggcaagagcggtgcaatggtgtgtaaaggtagctgagaagacgaaaagggcaagcatcttcctgctaccaggctggggaggcccaggcccacgaccccgaggagagggaacgcagggagactgaggtgacccttctttcccccggggcccggtcgtgtggttcggtgtctcttttctgttggacccttaccttgacccaggcgctgccggggcctgggcccgggctgcggcgcacggcactcccgggaggcagcgagactcgagttaggcccaacgcggcgccacggcgtttcctggccgggaatggcccgtacccgtgaggtgggggtggggggcagaaaaggcggagcgagcccgagcggggagggggagggccaggggcggagggggccggcactactgtgttggcggactggcgggactagggctgcgtgagtctctgagcgcaggcgggcggcggccgcccctcccccggcggcggcagcggcggcagcggcggcagctcactcagcccgctgcccgagcggaaacgccactgaccgcacggggattcccagtgccggcgccaggggcacgcgggacacgccccctcccgccgcgccattggcctctccgcccaccgccccacacttattggccggtgcgccgccaatcagcggaggctgccggggccgcctaaagaagaggctgtgctttggggctccggctcctcagagagcctcggctaggtaggggatcgggactctggcgggagggcggcttggtgcgtttgcggggatgggcggccgcggcaggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggagcgcttccggccgacgtctcgtcgctgattggcttcttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttctagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcagtggtgttccacatttgggcctattctcagtccagggagttttacaacaatagatgtattgagaatccaacctaaagcttaactttccactcccatgaatgcctctctcctttttctccatttataaactgagctattaaccattaatggttccaggtggatgtctcctccccatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagacttgtgggatacagaagaccaatgcagactttaatgtcttttctcttacactaagcaataaagaaataaaaattgaacttctagtatcctatttgtttaaactgctagctttacttaacttttgtgcttcatctatacaaagctgaaagctaagtctgcagccattactaaacatgaaagcaagtaatgataattttggatttcaaaaatgtagggccagagtttagccagccagtggtggtgcttgcctttatgcctttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttgggtcttttgacactgtgggctttctttaaagcctccttcctgccatgtggtctcttgtttgctactaacttcccatggcttaaatggcatggctttttgccttctaagggcagctgctgagatttgcagcctgatttccagggtggggttgggaaatctttcaaacactaaaattgtcctttaattttttttttaaaaaatgggttatataataaacctcataaaatagttatgaggagtgaggtggactaatattaaatgagtccctcccctataaaagagctattaaggctttttgtcttatacttaactttttttttaaatgtggtatctttagaaccaagggtcttagagttttagtatacagaaactgttgcatcgcttaatcagattttctagtttcaaatccagagaatccaaattcttcacagccaaagtcaaattaagaatttctgacttttaatgttaatttgcttactgtgaatataaaaatgatagcttttcctgaggcagggtctcactatgtatctctgcctgatctgcaacaagatatgtagactaaagttctgcctgcttttgtctcctgaatactaaggttaaaatgtagtaatacttttggaacttgcaggtcagattcttttataggggacacactaagggagcttgggtgatagttggtaaaatgtgtttcaagtgatgaaaacttgaattattatcaccgcaacctactttttaaaaaaaaaagccaggcctgttagagcatgcttaagggatccctaggacttgctgagcacacaagagtagttacttggcaggctcctggtgagagcatatttcaaaaaacaaggcagacaaccaagaaactacagttaaggttacctgtctttaaaccatctgcatatacacagggatattaaaatattccaaataatatttcattcaagttttcccccatcaaattgggacatggatttctccggtgaataggcagagttggaaactaaacaaatgttggttttgtgatttgtgaaattgttttcaagtgatagttaaagcccatgagatacagaacaaagctgctatttcgaggtctcttggtttatactcagaagcacttctttgggtttccctgcactatcctgatcatgtgctaggcctaccttaggctgattgttgttcaaataaacttaagtttcctgtcaggtgatgtcatatgatttcatatatcaaggcaaaacatgttatatatgttaaacatttgtacttaatgtgaaagttaggtctttgtgggtttgatttttaattttcaaaacctgagctaaataagtcatttttacatgtcttacatttggtggaattgtataattgtggtttgcaggcaagactctctgacctagtaaccctacctatagagcactttgctgggtcacaagtctaggagtcaagcatttcaccttgaagttgagacgttttgttagtgtatactagtttatatgttggaggacatgtttatccagaagatattcaggactatttttgactgggctaaggaattgattctgattagcactgttagtgagcattgagtggcctttaggcttgaattggagtcacttgtatatctcaaataatgctggccttttttaaaaagcccttgttctttatcaccctgttttctacataatttttgttcaaagaaatacttgtttggatctccttttgacaacaatagcatgttttcaagccatattttttttcctttttttttttttttttggtttttcgagacagggtttctctgtatagccctggctgtcctggaactcactttgtagaccaggctggcctcgaactcagaaatccgcctgcctctgcctcctgagtgccgggattaaaggcgtgcaccaccacgcctggctaagttggatattttgttatataactataaccaatactaactccactgggtggatttttaattcagtcagtagtcttaagtggtctttattggcccttcattaaaatctactgttcactctaacagaggctgttggtactagtggcacttaagcaacttcctacggatatactagcagattaagggtcagggatagaaactagtctagcgttttgtatacctaccagctttatactaccttgttctgatagaaatatttcaggacatctagagtgtactataaggttgatggtaagcttataaggaacttgaaagtggagtaactactccatttctctgaggggagaattaaaatttttgaccaagtgttgttgagccactgagaatggtctcagaacataacttcttaaggaaccttcccagattgccctcaacactgcaccacatttggtcctgcttgaacattgccatggctcttaaagtcttaattaagaatattaattgtgtaattattgtttttcctcctttagatcattccttgaggacaggacagtgcttgtttaaggctatatttctgctgtctgagcagcaacaggtcttcgagatcaacatgatgttcataatcccaagatgttgccatttatgttctcagaagcaagcagaggcatgatggtcagtgacagtaatgtcactgtgttaaatgttgctatgcagtttggatttttctaatgtagtgtaggtagaacatatgtgttctgtatgaattaaactcttaagttacaccttgtataatccatgcaatgtgttatgcaattaccattttaagtattgtagctttctttgtatgtgaggataaaggtgtttgtcataaaatgttttgaacatttccccaaagttccaaattataaaaccacaacgttagaacttatttatgaacaatggttgtagtttcatgcttttaaaatgcttaattattcaattaacaccgtttgtgttataatatatataaaactgacatgtagaagtgtttgtccagaacatttcttaaatgtatactgtctttagagagtttaatatagcatgtcttttgcaacatactaacttttgtgttggtgcgagcaatattgtgtagtcattttgaaaggagtcatttcaatgagtgtcagattgttttgaatgttattgaacattttaaatgcagacttgttcgtgttttagaaagcaaaactgtcagaagctttgaactagaaattaaaaagctgaagtatttcagaagggaaataagctacttgctgtattagttgaaggaaagtgtaatagcttagaaaatttaaaaccatatagttgtcattgctgaatatctggcagatgaaaagaaatactcagtggttcttttgagcaatataacagcttgttatattaaaaattttccccacagatataaactctaatctataactcataaatgttacaaatggatgaagcttacaaatgtggcttgacttgtcactgtgcttgttttagttatgtgaaagtttggcaataaacctatgtcctaaatagtcaaactgtggaatgactttttaatctattggtttgtctagaacagttatgttgccatttgccctaatggtgaaagaaaaagtggggagtgccttggcactgttcatttgtggtgtgaaccaaagaggggggcatgcacttacacttcaaacatccttttgaaagactgacaagtttgggtcttcacagttggaattgggcatcccttttgtcagggagggagggagggagggaggctggcttgttatgctgacaagtgtgattaaattcaaactttgaggtaagttggaggaacttgtacattgttaggagtgtgacaatttggactcttaatgatttggtcatacaaaatgaacctagaccaacttctggaagatgtatataataactccatgttacattgatttcacctgactaatacttatcccttatcaattaaatacagaagatgccagccatctgggccttttaacccagaaatttagtttcaaactcctaggttagtgttctcactgagctacatcctgatctagtcctgaaaataggaccaccatcacccccaaaaaaatctcaaataagatttatgctagtgtttcaaaattttaggaataggtaagattagaaagttttaaattttgagaaatggcttctctagaaagatgtacatagtgaacactgaatggctcctaaagagcctagaaaactggtactgagcacacaggactgagaggtctttcttgaaaagcatgtattgctttacgtgggtcacagaaggcaggcaggaagaacttgggctgaaactggtgtcttaagtggctaacatcttcacaactgatgagcaagaactttatcctgatgcaaaaaccatccaaacaaactaagtgaaaggtggcaatggatcccaggctgctctagaggaggacttgacttctcatcccatcacccacaccagatagctcatagactgccaattaacaccagcttctagcctccacaggcacctgcactggtacacataatttcacacaaacacagtaagaagccttccacctggcatggtattgcttatctttagttcccaacacttgggaggcagaggccagccagggctatgtgacaaaaaccttgtctagaggagaaacttcatagcttatttcctattcacgtaaccaggttagcaaaatttaccagccagagatgaagctaacagtgtccactatatttgtagtgttttaagtcaattttttaaatatacttaatagaattaaagctatggtgaaccaagtacaaacctggtgtattaacttgagaacttagcataaaaagtagttcatttgttcagtaaatattaaatgcttactggcaaagattatgtcaggaacttggtaaatggtgatgaaacaatcatagttgtacatcttggttctgtgatcaccttggtttgaggtaaaagtggttcctttgatcaaggatggaattttaagtttatattcaatcaataatgtattattttgtgattgcaaaattgcctatctagggtataaaacctttaaaaatttcataataccagttcattctccagttactaattccaaaaagccactgactatggtgccaatgtggattctgttctcaaaggaaggattgtctgtgccctttattctaatagaaacatcacactgaaaatctaagctgaaagaagccagactttcctaaataaataactttccataaagctcaaacaaggattacttttaggaggcactgttaaggaactgataagtaatgaggttacttatataatgatagtcccacaagactatctgaggaaaaatcagtacaactcgaaaacagaacaaccagctaggcaggaataacagggctcccaagtcaggaggtctatccaacacccttttctgttgagggccccagacctacatattgtatacaaacagggaggtgggtgattttaactctcctgaggtaccttggtaaatctttgtcctgagtaagcagtacagtgtacagtttacattttcatttaaagatacattagctccctctaccccctaagactgacaggcactttgggggtggggagggctttggaaaataacgcttccatacactaaaagagaaatttctttaattaggcttgttggttccatacatctactggtgtttctactacttagtaatattataatagtcacacaagcatctttgctctgtttaggttgtatatttattttaaggcagatgataaaactgtagatcttaagggatgcttctgcttctgagatgatacaaagaatttagaccataaaacagtaggttgcacaagcaatagaatatggcctaaagtgttctgacacttagaagccaagcagtgtaggcttcttaagaaataccattacaatcaccttgctagaaatcaagcattctggagtggtcaagcagtgtaacctgtactgtaagttacttttctgctatttttctcccaaagcaagttctttatgctgatatttccagtgttaggaactacaaatattaataagttgtcttcactcttttctttaccaaggagggtctcttccttcatcttgatctgaaggatgaacaaaggcttgagcagtgcgctttagaagataaactgcagcatgaaggcccccgatgttcacccagactacatggacctttcgccacacatgtcccattccagataaggcctggcacacacaaaaaacataagtcattaggctaccagtctgattctaaaacaacctaaaatcttcccacttaaatgctatgggtggtgggttggaaagttgactcagaaaatcacttgctgtttttagagaggatctgggttcagtttctgatacattgtggcttacaactataactccagttctagggggtccatccaacatcctcttctgttgagggcaccaaataaatgtattgtgtacaaacagggaggtgagtgatttaactctcgtgtatagtaccttggtaaaacatttcttgtcctgagtaagcagtacagctctgcctgtccctggtctacagacacggctcatttcccgaaggcaagctggatagagattccaatttctcttcttggatcccatcctataaaagaaggtcaagtttaatctattgcaaaaggtaaataggtagtttcttacatgagacaagaacaaatcttaggtgtgaagcagtcatcttttacaggccagagcctctattctatgccaatgaaggaaactgttagtccagtgttatagagttagtccagtgtatagttttctatcagaacactttttttttaaacaactgcaacttagcttattgaagacaaaccacgagtagaaatctgtccaagaagcaagtgcttctcagcctacaatgtggaataggaccatgtaatggtacagtgagtgaaatgaattatggcatgtttttctgactgagaagacagtacaataaaaggtaaactcatggtatttatttaaaaagaatccaatttctacctttttccaaatggcatatctgttacaataatatccacagaagcagttctcagtgggaggttgcagatatcccactgaacagcatcaatgggcaaaccccaggttgtttttctgtggagacaaaggtaagatatttcaatatattttcccaagctaatgagatggctcagcaaataatggtactggccattaagtctcatgacctgagcttgatcctcagggaccatgtggtacaaggagagacctaaatccttcagttggacttcaatcttctaccctcatgtccacacacaaataaatacaataaaaaacattctgcagtcgaatttctaaaagggcgaat

SEQ ID NO: 4 - DNA, 1073 bp, Mus musculus
  Description 5′ arm for Rosa26
caggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggagcgcttccggccgacgtctcgtcgctgattggcttcttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtct

SEQ ID NO: 5 - DNA, 4333 bp, Mus musculus
  Description 3′ arm for Rosa26
tagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcagtggtgttccacatttgggcctattctcagtccagggagttttacaacaatagatgtattgagaatccaacctaaagcttaactttccactcccatgaatgcctctctcctttttctccatttataaactgagctattaaccattaatggttccaggtggatgtctcctccccatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagacttgtgggatacagaagaccaatgcagactttaatgtcttttctcttacactaagcaataaagaaataaaaattgaacttctagtatcctatttgtttaaactgctagctttacttaacttttgtgcttcatctatacaaagctgaaagctaagtctgcagccattactaaacatgaaagcaagtaatgataattttggatttcaaaaatgtagggccagagtttagccagccagtggtggtgcttgcctttatgcctttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttgggtcttttgacactgtgggctttctttaaagcctccttcctgccatgtggtctcttgtttgctactaacttcccatggcttaaatggcatggctttttgccttctaagggcagctgctgagatttgcagcctgatttccagggtggggttgggaaatctttcaaacactaaaattgtcctttaattttttttttaaaaaatgggttatataataaacctcataaaatagttatgaggagtgaggtggactaatattaaatgagtccctcccctataaaagagctattaaggctttttgtcttatacttaactttttttttaaatgtggtatctttagaaccaagggtcttagagttttagtatacagaaactgttgcatcgcttaatcagattttctagtttcaaatccagagaatccaaattcttcacagccaaagtcaaattaagaatttctgacttttaatgttaatttgcttactgtgaatataaaaatgatagcttttcctgaggcagggtctcactatgtatctctgcctgatctgcaacaagatatgtagactaaagttctgcctgcttttgtctcctgaatactaaggttaaaatgtagtaatacttttggaacttgcaggtcagattcttttataggggacacactaagggagcttgggtgatagttggtaaaatgtgtttcaagtgatgaaaacttgaattattatcaccgcaacctactttttaaaaaaaaaagccaggcctgttagagcatgcttaagggatccctaggacttgctgagcacacaagagtagttacttggcaggctcctggtgagagcatatttcaaaaaacaaggcagacaaccaagaaactacagttaaggttacctgtctttaaaccatctgcatatacacagggatattaaaatattccaaataatatttcattcaagttttcccccatcaaattgggacatggatttctccggtgaataggcagagttggaaactaaacaaatgttggttttgtgatttgtgaaattgttttcaagtgatagttaaagcccatgagatacagaacaaagctgctatttcgaggtctcttggtttatactcagaagcacttctttgggtttccctgcactatcctgatcatgtgctaggcctaccttaggctgattgttgttcaaataaacttaagtttcctgtcaggtgatgtcatatgatttcatatatcaaggcaaaacatgttatatatgttaaacatttgtacttaatgtgaaagttaggtctttgtgggtttgatttttaattttcaaaacctgagctaaataagtcatttttacatgtcttacatttggtggaattgtataattgtggtttgcaggcaagactctctgacctagtaaccctacctatagagcactttgctgggtcacaagtctaggagtcaagcatttcaccttgaagttgagacgttttgttagtgtatactagtttatatgttggaggacatgtttatccagaagatattcaggactatttttgactgggctaaggaattgattctgattagcactgttagtgagcattgagtggcctttaggcttgaattggagtcacttgtatatctcaaataatgctggccttttttaaaaagcccttgttctttatcaccctgttttctacataatttttgttcaaagaaatacttgtttggatctccttttgacaacaatagcatgttttcaagccatattttttttcctttttttttttttttttggtttttcgagacagggtttctctgtatagccctggctgtcctggaactcactttgtagaccaggctggcctcgaactcagaaatccgcctgcctctgcctcctgagtgccgggattaaaggcgtgcaccaccacgcctggctaagttggatattttgttatataactataaccaatactaactccactgggtggatttttaattcagtcagtagtcttaagtggtctttattggcccttcattaaaatctactgttcactctaacagaggctgttggtactagtggcacttaagcaacttcctacggatatactagcagattaagggtcagggatagaaactagtctagcgttttgtatacctaccagctttatactaccttgttctgatagaaatatttcaggacatc

SEQ ID NO: 6 - DNA, 6039 bp, Artificial Sequence
  Description of Artificial Sequence Insert CAGGAS-creER
attgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagaagcgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacattgacatccactttgcctttctctccacaggtgtccactcccagggcggcctccggagcgatcgccggtccgcctaggctagccggccgcgtcgaccatgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggccgacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagtctgagcggccgaccggttcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcgccggtaaccgaagttcctatactttctagagaataggaacttcggaataggaacttcttaggtcaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcgatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctagatcccgcgccgaagttcctatactttctagagaataggaacttcggaataggaacttc

SEQ ID NO: 7 - DNA, 14411 bp, Artificial Sequence
  Description of Artificial Sequence targeting vector for Rosa26 locus with a CAGGS-creER insert
tttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgtcgagatctagatatcgatggccatagagttacgctagggataacagggtaatatagccgcggcaggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggagcgcttccggccgacgtctcgtcgctgattggcttcttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttctaggtaaccgatatccctgcaggttttcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagaagcgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacattgacatccactttgcctttctctccacaggtgtccactcccagggcggcctccggagcgatcgccggtccgcctaggctagccggccgcgtcgaccatgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggccgacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagtctgagcggccgaccggttcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcgccggtaaccgaagttcctatactttctagagaataggaacttcggaataggaacttcttaggtcaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcgatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctagatcccgcgccgaagttcctatactttctagagaataggaacttcggaataggaacttcaagcttaagcgctagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcagtggtgttccacatttgggcctattctcagtccagggagttttacaacaatagatgtattgagaatccaacctaaagcttaactttccactcccatgaatgcctctctcctttttctccatttataaactgagctattaaccattaatggttccaggtggatgtctcctccccatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagacttgtgggatacagaagaccaatgcagactttaatgtcttttctcttacactaagcaataaagaaataaaaattgaacttctagtatcctatttgtttaaactgctagctttacttaacttttgtgcttcatctatacaaagctgaaagctaagtctgcagccattactaaacatgaaagcaagtaatgataattttggatttcaaaaatgtagggccagagtttagccagccagtggtggtgcttgcctttatgcctttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttgggtcttttgacactgtgggctttctttaaagcctccttcctgccatgtggtctcttgtttgctactaacttcccatggcttaaatggcatggctttttgccttctaagggcagctgctgagatttgcagcctgatttccagggtggggttgggaaatctttcaaacactaaaattgtcctttaattttttttttaaaaaatgggttatataataaacctcataaaatagttatgaggagtgaggtggactaatattaaatgagtccctcccctataaaagagctattaaggctttttgtcttatacttaactttttttttaaatgtggtatctttagaaccaagggtcttagagttttagtatacagaaactgttgcatcgcttaatcagattttctagtttcaaatccagagaatccaaattcttcacagccaaagtcaaattaagaatttctgacttttaatgttaatttgcttactgtgaatataaaaatgatagcttttcctgaggcagggtctcactatgtatctctgcctgatctgcaacaagatatgtagactaaagttctgcctgcttttgtctcctgaatactaaggttaaaatgtagtaatacttttggaacttgcaggtcagattcttttataggggacacactaagggagcttgggtgatagttggtaaaatgtgtttcaagtgatgaaaacttgaattattatcaccgcaacctactttttaaaaaaaaaagccaggcctgttagagcatgcttaagggatccctaggacttgctgagcacacaagagtagttacttggcaggctcctggtgagagcatatttcaaaaaacaaggcagacaaccaagaaactacagttaaggttacctgtctttaaaccatctgcatatacacagggatattaaaatattccaaataatatttcattcaagttttcccccatcaaattgggacatggatttctccggtgaataggcagagttggaaactaaacaaatgttggttttgtgatttgtgaaattgttttcaagtgatagttaaagcccatgagatacagaacaaagctgctatttcgaggtctcttggtttatactcagaagcacttctttgggtttccctgcactatcctgatcatgtgctaggcctaccttaggctgattgttgttcaaataaacttaagtttcctgtcaggtgatgtcatatgatttcatatatcaaggcaaaacatgttatatatgttaaacatttgtacttaatgtgaaagttaggtctttgtgggtttgatttttaattttcaaaacctgagctaaataagtcatttttacatgtcttacatttggtggaattgtataattgtggtttgcaggcaagactctctgacctagtaaccctacctatagagcactttgctgggtcacaagtctaggagtcaagcatttcaccttgaagttgagacgttttgttagtgtatactagtttatatgttggaggacatgtttatccagaagatattcaggactatttttgactgggctaaggaattgattctgattagcactgttagtgagcattgagtggcctttaggcttgaattggagtcacttgtatatctcaaataatgctggccttttttaaaaagcccttgttctttatcaccctgttttctacataatttttgttcaaagaaatacttgtttggatctccttttgacaacaatagcatgttttcaagccatattttttttcctttttttttttttttttggtttttcgagacagggtttctctgtatagccctggctgtcctggaactcactttgtagaccaggctggcctcgaactcagaaatccgcctgcctctgcctcctgagtgccgggattaaaggcgtgcaccaccacgcctggctaagttggatattttgttatataactataaccaatactaactccactgggtggatttttaattcagtcagtagtcttaagtggtctttattggcccttcattaaaatctactgttcactctaacagaggctgttggtactagtggcacttaagcaacttcctacggatatactagcagattaagggtcagggatagaaactagtctagcgttttgtatacctaccagctttatactaccttgttctgatagaaatatttcaggacatctagcacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcc

SEQ ID NO: 8 - DNA, 18866 bp, Artificial Sequence
  Description of Artificial Sequence FABP-Cre targeting vector
tcgagacattcctgcgcatcgtgcagtgccgctctgtggagggcagctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattaagcttgatatcgaattctcagaatacaaaacagctttagggactgataaaatatatgtaaaattatgtttgtataataatagaaaaattgagaaataaacataaggctacgtgaaaaggttttgagttcaaaggtatttttctccacgtaatgcacatatacatgtgtgtttatatatgtgcatatgtatatgtagctacatgtgtataaatatattacatatacaagtgtgcacatgtataaacacatacatatgtacacataggtatatatgcatgtatgcattgctagagatgtgattcacatgtctgttgcacatatatacatctgtcaacatacatttcatgtatgcactcttatttcatgagcggtgataagacaccaaaaatgcaccatttacagagagctttgcccttgattggactcactaatgtttgctgaattagaacaaacttctgccttgcccattctgatttaaattagaacaaacttctgccttgcccattctgatttttatcgttagaacaaacttctgccttgcccattctgatttttatcgttagaacaaacttctgccttgcccattctgatttttatcgttagaacaaacttctgccttgcccattctgattttgatttttatcgttgaccattgctctcaggagttaatgtttgatcctggccataaagaaatcgacaatcactgacctatggcctatatttgaggaggaagaagccccttataaaatagccaacagtgggtggcctggcagacagagctgttgtggtcagggggatcaaataggccggcctaggcgcgtaagcgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacattgacatccactttgcctttctctccacaggtgtccactcccagggcggcctcgaccatgcccaagaagaagaggaaggtgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattagccattaacgcgtaaatgattgcagatccactagttctagggccgcgtcgacctcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcgccggtaaccgaagttcctatactttctagagaataggaacttcggaataggaacttcttaggtcaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcgatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctagatcccgcgccgaagttcctatactttctagagaataggaacttcggaataggaacttcaagcttaagcgctagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcagtggtgttccacatttgggcctattctcagtccagggagttttacaacaatagatgtattgagaatccaacctaaagcttaactttccactcccatgaatgcctctctcctttttctccatttataaactgagctattaaccattaatggttccaggtggatgtctcctccccatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagacttgtgggatacagaagaccaatgcagactttaatgtcttttctcttacactaagcaataaagaaataaaaattgaacttctagtatcctatttgtttaaactgctagctttacttaacttttgtgcttcatctatacaaagctgaaagctaagtctgcagccattactaaacatgaaagcaagtaatgataattttggatttcaaaaatgtagggccagagtttagccagccagtggtggtgcttgcctttatgcctttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttgggtcttttgacactgtgggctttctttaaagcctccttcctgccatgtggtctcttgtttgctactaacttcccatggcttaaatggcatggctttttgccttctaagggcagctgctgagatttgcagcctgatttccagggtggggttgggaaatctttcaaacactaaaattgtcctttaattttttttttaaaaaatgggttatataataaacctcataaaatagttatgaggagtgaggtggactaatattaaatgagtccctcccctataaaagagctattaaggctttttgtcttatacttaactttttttttaaatgtggtatctttagaaccaagggtcttagagttttagtatacagaaactgttgcatcgcttaatcagattttctagtttcaaatccagagaatccaaattcttcacagccaaagtcaaattaagaatttctgacttttaatgttaatttgcttactgtgaatataaaaatgatagcttttcctgaggcagggtctcactatgtatctctgcctgatctgcaacaagatatgtagactaaagttctgcctgcttttgtctcctgaatactaaggttaaaatgtagtaatacttttggaacttgcaggtcagattcttttataggggacacactaagggagcttgggtgatagttggtaaaatgtgtttcaagtgatgaaaacttgaattattatcaccgcaacctactttttaaaaaaaaaagccaggcctgttagagcatgcttaagggatccctaggacttgctgagcacacaagagtagttacttggcaggctcctggtgagagcatatttcaaaaaacaaggcagacaaccaagaaactacagttaaggttacctgtctttaaaccatctgcatatacacagggatattaaaatattccaaataatatttcattcaagttttcccccatcaaattgggacatggatttctccggtgaataggcagagttggaaactaaacaaatgttggttttgtgatttgtgaaattgttttcaagtgatagttaaagcccatgagatacagaacaaagctgctatttcgaggtctcttggtttatactcagaagcacttctttgggtttccctgcactatcctgatcatgtgctaggcctaccttaggctgattgttgttcaaataaacttaagtttcctgtcaggtgatgtcatatgatttcatatatcaaggcaaaacatgttatatatgttaaacatttgtacttaatgtgaaagttaggtctttgtgggtttgatttttaattttcaaaacctgagctaaataagtcatttttacatgtcttacatttggtggaattgtataattgtggtttgcaggcaagactctctgacctagtaaccctacctatagagcactttgctgggtcacaagtctaggagtcaagcatttcaccttgaagttgagacgttttgttagtgtatactagtttatatgttggaggacatgtttatccagaagatattcaggactatttttgactgggctaaggaattgattctgattagcactgttagtgagcattgagtggcctttaggcttgaattggagtcacttgtatatctcaaataatgctggccttttttaaaaagcccttgttctttatcaccctgttttctacataatttttgttcaaagaaatacttgtttggatctccttttgacaacaatagcatgttttcaagccatattttttttcctttttttttttttttttggtttttcgagacagggtttctctgtatagccctggctgtcctggaactcactttgtagaccaggctggcctcgaactcagaaatccgcctgcctctgcctcctgagtgccgggattaaaggcgtgcaccaccacgcctggctaagttggatattttgttatataactataaccaatactaactccactgggtggatttttaattcagtcagtagtcttaagtggtctttattggcccttcattaaaatctactgttcactctaacagaggctgttggtactagtggcacttaagcaacttcctacggatatactagcagattaagggtcagggatagaaactagtctagcgttttgtatacctaccagctttatactaccttgttctgatagaaatatttcaggacatctagagtgtactataaggttgatggtaagcttataaggaacttgaaagtggagtaactactccatttctctgaggggagaattaaaatttttgaccaagtgttgttgagccactgagaatggtctcagaacataacttcttaaggaaccttcccagattgccctcaacactgcaccacatttggtcctgcttgaacattgccatggctcttaaagtcttaattaagaatattaattgtgtaattattgtttttcctcctttagatcattccttgaggacaggacagtgcttgtttaaggctatatttctgctgtctgagcagcaacaggtcttcgagatcaacatgatgttcataatcccaagatgttgccatttatgttctcagaagcaagcagaggcatgatggtcagtgacagtaatgtcactgtgttaaatgttgctatgcagtttggatttttctaatgtagtgtaggtagaacatatgtgttctgtatgaattaaactcttaagttacaccttgtataatccatgcaatgtgttatgcaattaccattttaagtattgtagctttctttgtatgtgaggataaaggtgtttgtcataaaatgttttgaacatttccccaaagttccaaattataaaaccacaacgttagaacttatttatgaacaatggttgtagtttcatgcttttaaaatgcttaattattcaattaacaccgtttgtgttataatatatataaaactgacatgtagaagtgtttgtccagaacatttcttaaatgtatactgtctttagagagtttaatatagcatgtcttttgcaacatactaacttttgtgttggtgcgagcaatattgtgtagtcattttgaaaggagtcatttcaatgagtgtcagattgttttgaatgttattgaacattttaaatgcagacttgttcgtgttttagaaagcaaaactgtcagaagctttgaactagaaattaaaaagctgaagtatttcagaagggaaataagctacttgctgtattagttgaaggaaagtgtaatagcttagaaaatttaaaaccatatagttgtcattgctgaatatctggcagatgaaaagaaatactcagtggttcttttgagcaatataacagcttgttatattaaaaattttccccacagatataaactctaatctataactcataaatgttacaaatggatgaagcttacaaatgtggcttgacttgtcactgtgcttgttttagttatgtgaaagtttggcaataaacctatgtcctaaatagtcaaactgtggaatgactttttaatctattggtttgtctagaacagttatgttgccatttgccctaatggtgaaagaaaaagtggggagtgccttggcactgttcatttgtggtgtgaaccaaagaggggggcatgcacttacacttcaaacatccttttgaaagactgacaagtttgggtcttcacagttggaattgggcatcccttttgtcagggagggagggagggagggaggctggcttgttatgctgacaagtgtgattaaattcaaactttgaggtaagttggaggaacttgtacattgttaggagtgtgacaatttggactcttaatgatttggtcatacaaaatgaacctagaccaacttctggaagatgtatataataactccatgttacattgatttcacctgactaatacttatcccttatcaattaaatacagaagatgccagccatctgggccttttaacccagaaatttagtttcaaactcctaggttagtgttctcactgagctacatcctgatctagtcctgaaaataggaccaccatcacccccaaaaaaatctcaaataagatttatgctagtgtttcaaaattttaggaataggtaagattagaaagttttaaattttgagaaatggcttctctagaaagatgtacatagtgaacactgaatggctcctaaagagcctagaaaactggtactgagcacacaggactgagaggtctttcttgaaaagcatgtattgctttacgtgggtcacagaaggcaggcaggaagaacttgggctgaaactggtgtcttaagtggctaacatcttcacaactgatgagcaagaactttatcctgatgcaaaaaccatccaaacaaactaagtgaaaggtggcaatggatcccaggctgctctagaggaggacttgacttctcatcccatcacccacaccagatagctcatagactgccaattaacaccagcttctagcctccacaggcacctgcactggtacacataatttcacacaaacacagtaagaagccttccacctggcatggtattgcttatctttagttcccaacacttgggaggcagaggccagccagggctatgtgacaaaaaccttgtctagaggagaaacttcatagcttatttcctattcacgtaaccaggttagcaaaatttaccagccagagatgaagctaacagtgtccactatatttgtagtgttttaagtcaattttttaaatatacttaatagaattaaagctatggtgaaccaagtacaaacctggtgtattaacttgagaacttagcataaaaagtagttcatttgttcagtaaatattaaatgcttactggcaaagattatgtcaggaacttggtaaatggtgatgaaacaatcatagttgtacatcttggttctgtgatcaccttggtttgaggtaaaagtggttcctttgatcaaggatggaattttaagtttatattcaatcaataatgtattattttgtgattgcaaaattgcctatctagggtataaaacctttaaaaatttcataataccagttcattctccagttactaattccaaaaagccactgactatggtgccaatgtggattctgttctcaaaggaaggattgtctgtgccctttattctaatagaaacatcacactgaaaatctaagctgaaagaagccagactttcctaaataaataactttccataaagctcaaacaaggattacttttaggaggcactgttaaggaactgataagtaatgaggttacttatataatgatagtcccacaagactatctgaggaaaaatcagtacaactcgaaaacagaacaaccagctaggcaggaataacagggctcccaagtcaggaggtctatccaacacccttttctgttgagggccccagacctacatattgtatacaaacagggaggtgggtgattttaactctcctgaggtaccttggtaaatctttgtcctgagtaagcagtacagtgtacagtttacattttcatttaaagatacattagctccctctaccccctaagactgacaggcactttgggggtggggagggctttggaaaataacgcttccatacactaaaagagaaatttctttaattaggcttgttggttccatacatctactggtgtttctactacttagtaatattataatagtcacacaagcatctttgctctgtttaggttgtatatttattttaaggcagatgataaaactgtagatcttaagggatgcttctgcttctgagatgatacaaagaatttagaccataaaacagtaggttgcacaagcaatagaatatggcctaaagtgttctgacacttagaagccaagcagtgtaggcttcttaagaaataccattacaatcaccttgctagaaatcaagcattctggagtggtcaagcagtgtaacctgtactgtaagttacttttctgctatttttctcccaaagcaagttctttatgctgatatttccagtgttaggaactacaaatattaataagttgtcttcactcttttctttaccaaggagggtctcttccttcatcttgatctgaaggatgaacaaaggcttgagcagtgcgctttagaagataaactgcagcatgaaggcccccgatgttcacccagactacatggacctttcgccacacatgtcccattccagataaggcctggcacacacaaaaaacataagtcattaggctaccagtctgattctaaaacaacctaaaatcttcccacttaaatgctatgggtggtgggttggaaagttgactcagaaaatcacttgctgtttttagagaggatctgggttcagtttctgatacattgtggcttacaactataactccagttctagggggtccatccaacatcctcttctgttgagggcaccaaataaatgtattgtgtacaaacagggaggtgagtgatttaactctcgtgtatagtaccttggtaaaacatttcttgtcctgagtaagcagtacagctctgcctgtccctggtctacagacacggctcatttcccgaaggcaagctggatagagattccaatttctcttcttggatcccatcctataaaagaaggtcaagtttaatctattgcaaaaggtaaataggtagtttcttacatgagacaagaacaaatcttaggtgtgaagcagtcatcttttacaggccagagcctctattctatgccaatgaaggaaactgttagtccagtgttatagagttagtccagtgtatagttttctatcagaacactttttttttaaacaactgcaacttagcttattgaagacaaaccacgagtagaaatctgtccaagaagcaagtgcttctcagcctacaatgtggaataggaccatgtaatggtacagtgagtgaaatgaattatggcatgtttttctgactgagaagacagtacaataaaaggtaaactcatggtatttatttaaaaagaatccaatttctacctttttccaaatggcatatctgttacaataatatccacagaagcagttctcagtgggaggttgcagatgacctcgagggggggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgtcgagatctagatatcgatggccatagagttacgctagggataacagggtaatatagccgcggcaggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggagcgcttccggccgacgtctcgtcgctgattggcttcttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttctaggtaaccgatatccctgcaggggtgacctgcacgtctagggcgcagtagtccagggtttccttgatgatgtcatacttatcctgtcccttttttttccacagctcgcggttgaggacaaactcttcgcggtctttccagtactcctgcaggtgactgactgagtcgacttaattaaggccatagcggccatttctagacttagatccccgcgggatcttcaagcagacctacagcaagttcgacacaaactcacacaacgatgacgcactactcaagaactacgggctgctctactgcttcaggaaggacatggacaagg

SEQ ID NO: 9 - DNA, 21730 bp, Artificial Sequence
  Description of Artificial Sequence Cre substrate reporter
taaagggaacaaaagctgtcgagatctagatatcgatggccatagagttacgctagggataacagggtaatatagccgcggtcaggccctccgagcgtggtggagccgttctgtgagacagccggatcggcaggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggagcgcttccggccgacgtctcgtcgctgattggcttcttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttcctaggtaaccgatatccctgcaggttttcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagaagcgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacattgacatccactttgcctttctctccacaggtgtccactcccagggcggcctccggagcgatcgccggtccgcctaggctagccggccgcgtcgacttaattaaggccatagcggccattttcgacgacactgcagagacctacttcactaacaaccggtacagttcggtggagtacgcgcccggggagcccaagggcacgccctggcacccgcaccgcggcgaataacttcgtatagcatacattatacgaagttataagctcgatgaattctaccgggtaggggaggcgcttttcccaaggcagtctggagcatgcgctttagcagccccgctggcacttggcgctacacaagtggcctctggcctcgcacacattccacatccaccggtagcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcgaaggtcctcccgaggcccggcattctcgcacgcttcaaaagcgcacgtctgccgcgctgttctcctcttcctcatctccgggcctttcgacgatccagccgccaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtcgatgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagcttcagctgctcgactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccctacgcccccaactgagagaactcaaaggttaccccagttggggcactacggatccataacttcgtatagcatacattatacgaagttataccgggccaccggcgcgccatgcctaagaagaagaggaaggttgtcgcgagtagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggggggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgccagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctgcggccaaatcggccggcctaggcgcgccggtaaccgaagttcctatactttctagagaataggaacttcggaataggaacttcaagcttaagcgctagcactagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcagtggtgttccacatttgggcctattctcagtccagggagttttacaacaatagatgtattgagaatccaacctaaagcttaactttccactcccatgaatgcctctctcctttttctccatttataaactgagctattaaccattaatggttccaggtggatgtctcctccccatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagacttgtgggatacagaagaccaatgcagactttaatgtcttttctcttacactaagcaataaagaaataaaaattgaacttctagtatcctatttgtttaaactgctagctttacttaacttttgtgcttcatctatacaaagctgaaagctaagtctgcagccattactaaacatgaaagcaagtaatgataattttggatttcaaaaatgtagggccagagtttagccagccagtggtggtgcttgcctttatgcctttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttgggtcttttgacactgtgggctttctttaaagcctccttcctgccatgtggtctcttgtttgctactaacttcccatggcttaaatggcatggctttttgccttctaagggcagctgctgagatttgcagcctgatttccagggtggggttgggaaatctttcaaacactaaaattgtcctttaattttttttttaaaaaatgggttatataataaacctcataaaatagttatgaggagtgaggtggactaatattaaatgagtccctcccctataaaagagctattaaggctttttgtcttatacttaactttttttttaaatgtggtatctttagaaccaagggtcttagagttttagtatacagaaactgttgcatcgcttaatcagattttctagtttcaaatccagagaatccaaattcttcacagccaaagtcaaattaagaatttctgacttttaatgttaatttgcttactgtgaatataaaaatgatagcttttcctgaggcagggtctcactatgtatctctgcctgatctgcaacaagatatgtagactaaagttctgcctgcttttgtctcctgaatactaaggttaaaatgtagtaatacttttggaacttgcaggtcagattcttttataggggacacactaagggagcttgggtgatagttggtaaaatgtgtttcaagtgatgaaaacttgaattattatcaccgcaacctactttttaaaaaaaaaagccaggcctgttagagcatgcttaagggatccctaggacttgctgagcacacaagagtagttacttggcaggctcctggtgagagcatatttcaaaaaacaaggcagacaaccaagaaactacagttaaggttacctgtctttaaaccatctgcatatacacagggatattaaaatattccaaataatatttcattcaagttttcccccatcaaattgggacatggatttctccggtgaataggcagagttggaaactaaacaaatgttggttttgtgatttgtgaaattgttttcaagtgatagttaaagcccatgagatacagaacaaagctgctatttcgaggtctcttggtttatactcagaagcacttctttgggtttccctgcactatcctgatcatgtgctaggcctaccttaggctgattgttgttcaaataaacttaagtttcctgtcaggtgatgtcatatgatttcatatatcaaggcaaaacatgttatatatgttaaacatttgtacttaatgtgaaagttaggtctttgtgggtttgatttttaattttcaaaacctgagctaaataagtcatttttacatgtcttacatttggtggaattgtataattgtggtttgcaggcaagactctctgacctagtaaccctacctatagagcactttgctgggtcacaagtctaggagtcaagcatttcaccttgaagttgagacgttttgttagtgtatactagtttatatgttggaggacatgtttatccagaagatattcaggactatttttgactgggctaaggaattgattctgattagcactgttagtgagcattgagtggcctttaggcttgaattggagtcacttgtatatctcaaataatgctggccttttttaaaaagcccttgttctttatcaccctgttttctacataatttttgttcaaagaaatacttgtttggatctccttttgacaacaatagcatgttttcaagccatattttttttcctttttttttttttttttggtttttcgagacagggtttctctgtatagccctggctgtcctggaactcactttgtagaccaggctggcctcgaactcagaaatccgcctgcctctgcctcctgagtgccgggattaaaggcgtgcaccaccacgcctggctaagttggatattttgttatataactataaccaatactaactccactgggtggatttttaattcagtcagtagtcttaagtggtctttattggcccttcattaaaatctactgttcactctaacagaggctgttggtactagtggcacttaagcaacttcctacggatatactagcagattaagggtcagggatagaaactagtctagcgttttgtatacctaccagctttatactaccttgttctgatagaaatatttcaggacatctagagtgtactataaggttgatggtaagcttataaggaacttgaaagtggagtaactactccatttctctgaggggagaattaaaatttttgaccaagtgttgttgagccactgagaatggtctcagaacataacttcttaaggaaccttcccagattgccctcaacactgcaccacatttggtcctgcttgaacattgccatggctcttaaagtcttaattaagaatattaattgtgtaattattgtttttcctcctttagatcattccttgaggacaggacagtgcttgtttaaggctatatttctgctgtctgagcagcaacaggtcttcgagatcaacatgatgttcataatcccaagatgttgccatttatgttctcagaagcaagcagaggcatgatggtcagtgacagtaatgtcactgtgttaaatgttgctatgcagtttggatttttctaatgtagtgtaggtagaacatatgtgttctgtatgaattaaactcttaagttacaccttgtataatccatgcaatgtgttatgcaattaccattttaagtattgtagctttctttgtatgtgaggataaaggtgtttgtcataaaatgttttgaacatttccccaaagttccaaattataaaaccacaacgttagaacttatttatgaacaatggttgtagtttcatgcttttaaaatgcttaattattcaattaacaccgtttgtgttataatatatataaaactgacatgtagaagtgtttgtccagaacatttcttaaatgtatactgtctttagagagtttaatatagcatgtcttttgcaacatactaacttttgtgttggtgcgagcaatattgtgtagtcattttgaaaggagtcatttcaatgagtgtcagattgttttgaatgttattgaacattttaaatgcagacttgttcgtgttttagaaagcaaaactgtcagaagctttgaactagaaattaaaaagctgaagtatttcagaagggaaataagctacttgctgtattagttgaaggaaagtgtaatagcttagaaaatttaaaaccatatagttgtcattgctgaatatctggcagatgaaaagaaatactcagtggttcttttgagcaatataacagcttgttatattaaaaattttccccacagatataaactctaatctataactcataaatgttacaaatggatgaagcttacaaatgtggcttgacttgtcactgtgcttgttttagttatgtgaaagtttggcaataaacctatgtcctaaatagtcaaactgtggaatgactttttaatctattggtttgtctagaacagttatgttgccatttgccctaatggtgaaagaaaaagtggggagtgccttggcactgttcatttgtggtgtgaaccaaagaggggggcatgcacttacacttcaaacatccttttgaaagactgacaagtttgggtcttcacagttggaattgggcatcccttttgtcagggagggagggagggagggaggctggcttgttatgctgacaagtgtgattaaattcaaactttgaggtaagttggaggaacttgtacattgttaggagtgtgacaatttggactcttaatgatttggtcatacaaaatgaacctagaccaacttctggaagatgtatataataactccatgttacattgatttcacctgactaatacttatcccttatcaattaaatacagaagatgccagccatctgggccttttaacccagaaatttagtttcaaactcctaggttagtgttctcactgagctacatcctgatctagtcctgaaaataggaccaccatcacccccaaaaaaatctcaaataagatttatgctagtgtttcaaaattttaggaataggtaagattagaaagttttaaattttgagaaatggcttctctagaaagatgtacatagtgaacactgaatggctcctaaagagcctagaaaactggtactgagcacacaggactgagaggtctttcttgaaaagcatgtattgctttacgtgggtcacagaaggcaggcaggaagaacttgggctgaaactggtgtcttaagtggctaacatcttcacaactgatgagcaagaactttatcctgatgcaaaaaccatccaaacaaactaagtgaaaggtggcaatggatcccaggctgctctagaggaggacttgacttctcatcccatcacccacaccagatagctcatagactgccaattaacaccagcttctagcctccacaggcacctgcactggtacacataatttcacacaaacacagtaagaagccttccacctggcatggtattgcttatctttagttcccaacacttgggaggcagaggccagccagggctatgtgacaaaaaccttgtctagaggagaaacttcatagcttatttcctattcacgtaaccaggttagcaaaatttaccagccagagatgaagctaacagtgtccactatatttgtagtgttttaagtcaattttttaaatatacttaatagaattaaagctatggtgaaccaagtacaaacctggtgtattaacttgagaacttagcataaaaagtagttcatttgttcagtaaatattaaatgcttactggcaaagattatgtcaggaacttggtaaatggtgatgaaacaatcatagttgtacatcttggttctgtgatcaccttggtttgaggtaaaagtggttcctttgatcaaggatggaattttaagtttatattcaatcaataatgtattattttgtgattgcaaaattgcctatctagggtataaaacctttaaaaatttcataataccagttcattctccagttactaattccaaaaagccactgactatggtgccaatgtggattctgttctcaaaggaaggattgtctgtgccctttattctaatagaaacatcacactgaaaatctaagctgaaagaagccagactttcctaaataaataactttccataaagctcaaacaaggattacttttaggaggcactgttaaggaactgataagtaatgaggttacttatataatgatagtcccacaagactatctgaggaaaaatcagtacaactcgaaaacagaacaaccagctaggcaggaataacagggctcccaagtcaggaggtctatccaacacccttttctgttgagggccccagacctacatattgtatacaaacagggaggtgggtgattttaactctcctgaggtaccttggtaaatctttgtcctgagtaagcagtacagtgtacagtttacattttcatttaaagatacattagctccctctaccccctaagactgacaggcactttgggggtggggagggctttggaaaataacgcttccatacactaaaagagaaatttctttaattaggcttgttggttccatacatctactggtgtttctactacttagtaatattataatagtcacacaagcatctttgctctgtttaggttgtatatttattttaaggcagatgataaaactgtagatcttaagggatgcttctgcttctgagatgatacaaagaatttagaccataaaacagtaggttgcacaagcaatagaatatggcctaaagtgttctgacacttagaagccaagcagtgtaggcttcttaagaaataccattacaatcaccttgctagaaatcaagcattctggagtggtcaagcagtgtaacctgtactgtaagttacttttctgctatttttctcccaaagcaagttctttatgctgatatttccagtgttaggaactacaaatattaataagttgtcttcactcttttctttaccaaggagggtctcttccttcatcttgatctgaaggatgaacaaaggcttgagcagtgcgctttagaagataaactgcagcatgaaggcccccgatgttcacccagactacatggacctttcgccacacatgtcccattccagataaggcctggcacacacaaaaaacataagtcattaggctaccagtctgattctaaaacaacctaaaatcttcccacttaaatgctatgggtggtgggttggaaagttgactcagaaaatcacttgctgtttttagagaggatctgggttcagtttctgatacattgtggcttacaactataactccagttctagggggtccatccaacatcctcttctgttgagggcaccaaataaatgtattgtgtacaaacagggaggtgagtgatttaactctcgtgtatagtaccttggtaaaacatttcttgtcctgagtaagcagtacagctctgcctgtccctggtctacagacacggctcatttcccgaaggcaagctggatagagattccaatttctcttcttggatcccatcctataaaagaaggtcaagtttaatctattgcaaaaggtaaataggtagtttcttacatgagacaagaacaaatcttaggtgtgaagcagtcatcttttacaggccagagcctctattctatgccaatgaaggaaactgttagtccagtgttatagagttagtccagtgtatagttttctatcagaacactttttttttaaacaactgcaacttagcttattgaagacaaaccacgagtagaaatctgtccaagaagcaagtgcttctcagcctacaatgtggaataggaccatgtaatggtacagtgagtgaaatgaattatggcatgtttttctgactgagaagacagtacaataaaaggtaaactcatggtatttatttaaaaagaatccaatttctacctttttccaaatggcatatctgttacaataatatccacagaagcagttctcagtgggaggttgcagatgacctcgagggggggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcac